(19) United States
(12) Patent Application Publication
(10) Pub. No.: US 2013/0288255 A1
(43) Pub. Date: Oct. 31, 2013

(54) BIOMARKERS OF AGEING

(71) Applicants: Henri Martens, Saint-Nicolas (BE); Marie De Saint-Hubert, Walhain (BE); Thi Kim Duy Vo, Namur (BE); Gabriel Morrhaye, Marneffe (BE); Olivier Toussaint, Vedrin (BE)

(72) Inventors: Henri Martens, Saint-Nicolas (BE); Marie De Saint-Hubert, Walhain (BE); Thi Kim Duy Vo, Namur (BE); Gabriel Morrhaye, Marneffe (BE); Olivier Toussaint, Vedrin (BE)

(73) Assignee: Universite De Liege Universite Catholique De Louvain

(21) Appl. No.: 13/522,241

(22) PCT Filed: Jan. 14, 2011
(86) PCT No.: PCT/EP11/50472

§ 371 Date: Jul. 13, 2012

(30) Foreign Application Priority Data
Jan. 15, 2010 (EP) PCT/EP10/50486

(51) Int. Cl. C12Q 1/68

(52) U.S. Cl. CPC C12Q 1/6883
USPC 435/6.12

(57) ABSTRACT

The present invention relates to methods to predict the functional decline of a patient (preferable an elder patient).

### FIELD OF THE INVENTION

The present invention relates to the characterization and early identification of frailty and/or of functional decline in patients, especially in elder patients

### BACKGROUND OF THE INVENTION AND STATE OF THE ART

Frailty is a major public health issue to provide adapted care to elders both in the community and in the hospital.

Frailty is an age-related vulnerability to stressing events, due to multisystemic reduction of the adaptative reserve, predicting adverse outcomes and associated to lower recovery.

At a clinical level, frailty is a multidimensional condition, including physical but also psychological, cognitive and social domains.

Adverse outcomes include functional decline, increased health care, utilization, geriatric syndromes, institutionalization, hospitalization and mortality.

Life course approach proposed psychosocial, environmental, diseases, molecular and cellular determinants leading to decline in physiological reserves, clinical frailty and finally to adverse health outcomes.

Functional decline (FD) may be defined as the loss of ability to perform some activities of daily living (ADL) i.e. to wash, dress or walk. It frequently occurs after hospitalization of older patients, due not only to illness severity but also to their premorbid status. Such decline is associated with higher health care service use, institutionalisation and death. Several authors have suggested that the extent of FD after acute stress reflects the level of frailty, as “inability to withstand acute illness without loss of function”.

Identification of elderly at risk of functional decline is a major public health concern since targeted geriatric interventions may prevent or limit functional loss, as well in community-dwelling subjects as in hospitalized patients.

In the hospital setting, several screening tools predicting functional decline after hospitalization have been proposed (Cornette et al. Eur. J. Public Health 16:203-8, 2006). A recent systematic review compared several of these tools and, since none had sufficient predictive validity, adjunctive procedures are needed to improve the predictive value of these instruments.

Indeed, research on hospitalised aged persons raise several methodological problems and application of genes expression analysis in a hospital-based study face several challenges: technical expertise, quantity of available blood and frequency of sample collection, infrastructure and coordination between clinicians and biologists, development of tools facilitating storage, transmission and analysis of the amount of generated data. These factors explain the need for interdisciplinary expertise.

A Telomere is a region of repetitive DNA at the end of a chromosome, which protects the end of the chromosome from deterioration. The telomeres are consumed during cell division and replenished by the enzyme telomerase reverse transcriptase.

The telomere shortening mechanism is believed to limit certain cell types to a fixed number of divisions. Animal studies suggest that this mechanism is responsible for aging at the cellular level.

### SUMMARY OF THE INVENTION

The present invention is based on the observation that frailty of a patient can be predicted based on biological markers. More particularly it has been found that frailty can be predicted based on a measurement of telomere length of PBMC present in a blood sample of the patient. Accordingly, a first aspect of the present invention relates to a method to predict the functional decline of a patient (preferably an elder patient) comprising or consisting of the steps of:
Measuring the telomere length of PBMC from a blood sample obtained from said patient, preferably by measuring the T/S ratio; and
deducing from said telomere length, whether the said patient is likely to have a functional decline.

In particular embodiments, the methods of the invention further comprise one or more of the following steps:
determining the functional status of a patient
measuring cytokine content of the said blood sample and/or cytokine produced by PBMC present in said blood sample;
measuring sj and dβ TREC in PBMC present in the said blood sample;
   and the step of deducing whether the said patient is likely to have a functional decline is further based on one or more of said criteria.

Preferably, in the method of the invention, the telomere length is measured by quantitative PCR.

In particular embodiments of the methods described herein, the telomere length is measured by a method developed by the present inventors, which is described more in detail herein below.

In particular embodiments of the methods of the invention, a reduced telomere length (possibly measured by T/S ratio) represents a worse prognosis.

A further aspect of the present invention relates to a method to predict the functional decline of a patient (preferably an elder patient) comprising or consisting of the steps of:
measuring cytokine content of the said blood sample and/or cytokine produced by PBMC present in a blood sample of said patient;
deducing from said cytokine content whether the said patient is likely to have a functional decline.

In particular embodiments, the methods of the present invention comprise quantification of cytokines present in a blood sample or in stimulated PBMC-conditioned medium.

The preferred cytokines measured in the method of the invention are 1, 2, 3, 4 or all the cytokines selected from the group consisting of, TNF-α, IFN-γ, IL-4, IL-6 and IGF1.

Advantageously, in particular embodiments of the methods of the invention (the protein content of) IL-6 and IGF1 are measured.

In particular embodiments of the methods of the invention, a high IL-6 content represents a worse prognosis.

In further particular embodiments of the method of the invention, a high IGF1 content represents a good prognosis.

Yet a further aspect of the present invention relates to methods to predict the functional decline of a patient (preferably an elder patient) comprising or consisting of the steps of:
measuring sj and dβ TREC in PBMC present in a blood sample of said patient;
deducing, from said sj and dβ TREC values, whether the said patient is likely to have a functional decline.

In particular embodiments, the methods of the present invention comprise the quantification of sjTREC and DJβTREC, preferably by quantitative PCR.

In particular embodiments of the methods of the invention, a low sjTREC/DJβTREC ratio represent a worse prognosis.

Yet a further aspect of the present invention relates to methods to predict the functional decline of a patient (preferably an elder patient) comprising or consisting of the steps of:
measuring the number of CD4-positive cells in a sample of said patient
deducing from said number of CD4-positive cells whether the said patient is likely to have a functional decline.

In particular embodiments the methods of the present invention comprise the quantification of CD4-positive cells in a blood sample.

Advantageously, in particular embodiments of the methods of the invention CD8-positive cells are further also measured and/or quantified.

Advantageously, in particular embodiments of the methods of the invention further CD4CD28 positive cells are measured.

Advantageously, in particular embodiments of the methods of the invention further CD8CD28 positive cells are measured.

Yet a further aspect of the present invention relates to methods to predict the functional decline of a patient (preferably an elder patient) comprising or consisting of the steps of:
measuring, in a sample of said patient, the expression of one or more genes selected from the group consisting of ALDOA, BCL2, CASP8, CCL5, CCNH, CD28, CD69, CLU, CTSD, CTSH, CTSS, CTSZ, DDIT3, DNAJB1, E2F5, EGR3, EIF4A1, FAS, FASLG, FOS, GAA, GAPDH, GPX1, GRB2, HBEGF, HDAC2, HK1, HMOX1, HSP90AA1, HSPA4, HSPA6, HSPB1, IFNG, IGF1R, IGFBP3, IL10RB, IL11RA, IL15, IL1B, IL1R1, IL1R2, IL1RN, IL4R, IL8, IRAK-M, LCK, MAP2K1, MAP2K2, MAPK9, MAX, MDH1, MYBCBP, PPIE, PRDX6, PSMA2, PSMB8, PSMB9, PSMC6, PSMD1, PSMD11, PSMD12, PTGS1, PTGS2, RPL13A, RPS9, SERPINB2, SERPINE1, SOCS1, SOCS3, SOD2, SRI, TFRC, TGFA, TGFB1, TIMP1, TIMP2, TLR4, TNFRSF1A, TNFRSF1B and UBE2V1; and
deducing from said expression whether the said patient is likely to have a functional decline.

In particular embodiments, the methods of the present invention comprise the quantification of 1, 2, 3, 4, 5, 6, 7, 8, 9, 10 or all the genes selected from the group consisting of SERPINB2, IL1R1, IL1RN, TIMP2, MAP2K2, GAPDH, MAPK9, CASP8, LCK, BCL2, CD28, IGFBP3, TGFB1, IL15, IL10RB, MDH1, PSMA2, CCL5, PSMD11, RPL13A, IL4R, DNAJB1 and CTSZ. In further particular embodiments quantification is ensured by quantitative PCR of the mRNA of the one or more genes, obtained from PBMC present in a sample, such as the blood sample, of said patient.

Advantageously, in particular embodiments of the methods of the invention, the expression of SERPINB2, IL1R1 and CASP8 are measured.

Advantageously, in particular embodiments of the methods of the invention, the expression TGFB1, IL15, RPL13A and IL4R are measured.

Advantageously, in particular embodiments of the methods of the invention, the expression of 1, 2, 3, 4, 5, 6, 7, 8, 9, 10 or all the genes selected from the group consisting of SERPINB2, IL1R1, IL1RN, TIMP2, MAP2K2, GAPDH, MAPK9, CASP8, LCK, BCL2, CD28 is measured, preferably by quantitative PCR of their mRNA obtained from PBMC present in the blood sample.

The methods described herein may further comprise the step of determining the functional status of a patient. Preferably, in the methods of the invention, the functional status of a patient is determined by methods selected from the group consisting of ADL, iADL, HARP, ISAR and SHERPA, being preferably SHERPA.

In a further aspect of the present invention, kits are provided for use in determining frailty or likeliness of decline in a patient, based on analysis of one or more samples of said patient.

In particular embodiments the kit to determine frailty comprises or consists of means to measure telomere length. More particularly, the kit comprises instructions for determining frailty based on the measured telomere length.

In particular embodiments of the invention, kits are provided for determining frailty in a patient, wherein the kit comprises, in addition to means to measure telomere length, means to measure sjTREC and DβTREC, and/or means to measure cytokine content and/or means to measure the expression of one or more specific genes.

In further particular embodiments, a kit is provided comprising:
Means to measure telomere length;
Optionally means to measure sjTREC and DβTREC;
Optionally means to measure cytokine content;
Optionally means to measure mRNA content.

In particular embodiments, the kit of the invention comprises specific primers able to amplify by PCR the whole telomere and a reference single-copy gene (S) (e.g., CD3) generating a signal proportional to the average telomere length and, optionally, buffers and reagents for quantitative PCR, preferably Real-Time quantitative PCR.

In further particular embodiments, the kit of the invention further comprises means to measure (quantify) cytokine content comprise antibodies specifically recognizing said cytokines.

In further particular embodiments, in the kit of the invention, the means to measure (quantify) cytokine content are means for ELISA.

In further particular embodiments, the kit of the invention comprises means to measure (quantify) cytokines selected from the group consisting of TNF-α, IFN-γ IL-4, IL-6 and IGF1.

In further particular embodiments, the kit of the invention comprises means to measure both IL-6 and IGF1.

In further particular embodiments, the kit of the invention further comprises means to measure (quantify) the mRNA of 1, 2, 3, 4, 5, 6, 7, 8, 9, 10 or all the mRNA selected from the group consisting of SERPINB2, IL1R1, IL1RN, TIMP2, MAP2K2, GAPDH, MAPK9, CASP8, LCK, BCL2, CD28, IGFBP3, TGFB1, IL15, IL10RB, MDH1, PSMA2, CCL5, PSMD11, RPL13A, IL4R, DNAJB1 and CTSZ.

In a further aspect, the present invention relates to a protein (set) of 1, 2, 3, 4, 5, 6, 7, 8, 9, 10 or all the proteins of table 13, and to tools for detecting expression of the protein set in a sample.

In particular embodiments, the set of the invention comprises 1, 2, 3, 4, 5, 6, 7, 8, 9, 10 or all the proteins of table 13 over-expressed in frailty patients and 1, 2, 3, 4, 5, 6, 7, 8, 9, 10 or all the proteins of table 13 under-expressed in frailty patients.

The protein set or said tools are advantageously provided on a solid support.

In particular embodiments, the present invention provides means such as specific antibodies to recognize the protein (set) of the present invention.

In a particular embodiment, the present invention provides a gene (set) comprising 1, 2, 3, 4, 5, 6, 7; 8, 9, 10 or all the genes selected from the group consisting of ALDOA, BCL2, CASP8, CCL5, CCNH, CD28, CD69, CLU, CTSD, CTSH, CTSS, CTSZ, DDIT3, DNAJB1, E2F5, EGR3, EIF4A1, FAS, FASLG, FOS, GAA, GAPDH, GPX1, GRB2, HBEGF, HDAC2, HK1, HMOX1, HSP90AA1, HSPA4, HSPA6, HSPB1, IFNG, IGF1R, IGFBP3, IL10RB, IL11RA, IL15, IL1B, IL1R1, IL1R2, IL1RN, IL4R, IL8, IRAK-M, LCK, MAP2K1, MAP2K2, MAPK9, MAX, MDH1, MYBCBP, PPIE, PRDX6, PSMA2, PSMB8, PSMB9, PSMC6, PSMD1, PSMD11, PSMD12, PTGS1, PTGS2, RPL13A, RPS9, SERPINB2, SERPINE1, SOCS1, SOCS3, SOD2, SRI, TFRC, TGFA, TGFB1, TIMP1, TIMP2, TLR4, TNFRSF1A, TNFRSF1B and UBE2V1, or means for detecting said genes, being preferably on a solid support.

In particular embodiments, the most preferred genes of the gene set of the invention are 1, 2, 3, 4, 5, 6, 7, 8, 9, 10 or all the mRNA selected from the group consisting of SERPINB2, IL1R1, IL1RN, TIMP2, MAP2K2, GAPDH, MAPK9, CASP8, LCK, BCL2, CD28, IGFBP3, TGFB1, IL15, IL10RB, MDH1, PSMA2, CCL5, PSMD11, RPL13A, IL4R, DNAJB1 and CTSZ.

Another aspect of the present invention is related to methods and means to calibrate the measurement of the length of telomeres.

In particular embodiments, the methods for calibrating the measurement of telomere length comprise:
amplifying and quantifying the complete telomere using specific primers;
amplifying and quantifying a reference single-copy gene (S) in the same sample;
comparing said length with a standard comprising 1, 2, 3, 4 or more DNA fragments (possibly (each) on a plasmid) (each) in a known concentration encoding one single-copy gene (S) (or fragment thereof) and several telomeric repeats of different sizes (T).

Means are also provided to measure the length of telomeres. In particular embodiments, these means comprise:
one or more specific primers and optionally a buffer to amplify and possibly to quantify the complete telomere (T); and optionally one or more of the following,
specific primers and optionally a buffer to amplify and possibly to quantify a single-copy gene (S);
1, 2, 3, 4 or more DNA fragment (possibly (each) on a plasmid) (each) in a known concentration encoding one single-copy gene (S) (or fragment thereof) and several telomeric repeats of different size (T).

In particular embodiments, the calibrator used in the calibration of the measurement of telomere length in the methods of the present invention corresponds to a set of plasmids which can be described as follows: plasmid n° 1 containing one or more copy of the selected reference single-copy gene S and about 10 successive copies of a telomeric repeat T, plasmid n° 2 containing one or more copy of the selected reference single-copy gene S and about 20 successive copies of the same telomeric repeat, plasmid n° 3 containing one or more copy of the selected reference single-copy gene S and about 30 successive copies of the telomeric repeat . . . etc, plasmid n° n containing one or more copy of the selected reference single-copy gene S and about 10×n successive copies of the telomeric repeat. More particularly, an appropriate amount of each of these plasmids is used in a PCR reaction, said amount being known or estimated from the total DNA quantity in the sample to be characterized.

A further aspect of the invention provides for methods for screening for compounds capable of reducing the risk for functional decline, which are based on testing whether or not the compounds are capable of causing a change in telomere length of PBMCs from a blood sample obtained from patients at risk of functional decline.

In particular embodiments, the methods of the invention comprise the steps of:

Measuring the telomere length of PBMC from a blood sample obtained from patients, wherein each of said patients

has been previously identified as having a risk of functional decline based on average telomere length of PBMCs from a blood sample; and

has been administered a compound of interest; and

deducing from said telomere length, whether the compound administered to said patient has resulted in a reduced risk of functional decline.

More particularly, the methods of the invention are methods for determining the effect of a compound on the risk of functional decline of a patient. In particular embodiments, the methods of the invention comprise:

Measuring the telomere length of PBMC from a blood sample obtained from a patient prior to administration of a compound of interest;

Measuring the telomere length of PBMC from a blood sample obtained from said patient after administration of said compound of interest;
comparing the values of telomere length and deducing therefrom whether the compound has an effect on the risk of functional decline of said patient.

In particular embodiments of the methods of the present invention, telomere length is determined by measuring the T/S ratio. In further particular embodiments of the methods of the invention, an increased telomere length is indicative of the fact that the compound is capable of reducing the risk of functional decline of a patient.

Similarly, the present invention provides methods for determining the therapeutic effect of a compound on a patient, which are based on measuring the effect of said compound on the risk of functional decline of said patient. Such methods may comprise the steps of:

Measuring the telomere length of PBMC from a blood sample obtained from a patient prior to administration of a compound of interest;

Measuring the telomere length of PBMC from a blood sample obtained from said patient after administration of said compound of interest;

comparing the values of telomere length and deducing therefrom whether the compound has a therapeutic effect, based on whether or not the compound has an effect on the risk of functional decline of said patient.

Accordingly, the present invention provides tools for use in screening methods and methods for the determination of therapeutic effects of compounds.

A further aspect of the invention provides computer program products for enabling a computer to predict based on values obtained from a PBMC sample of a patient, the functional decline of a patient, comprising:

a computer readable medium, and

software instructions, on the computer-readable medium for enabling the computer to perform predetermined operations comprising determining the value of telomere length in said sample, most particularly based on determining the T/S value in a sample of a patient. Similarly, the invention provides computer program products for enabling a computer to determine the effect of a compound on the risk of functional decline of a patient, which programs involve comparisons based on values of telomere lengths determined in different samples as described hereinabove.

### BRIEF DESCRIPTION OF THE FIGURES AND TABLES

The invention will now be described, inter alia with reference to the accompanying Figures, which are provided by way of example only and should not be considered to limit the scope of the present invention.

FIG. 1: sj and DβTREC Quantification

A. sjTREC frequency B. DβTREC frequency C. Intrathymic proliferation
Box-and-whisker plots of log 10 for each populations: Community Dwelling (CD), Non Functional Decline (NFD) and Functional Decline (FD). Each box contains the middle 50% of the data, the horizontal line within each box represents the median, and the whiskers extend out to the 10th and 90th percentiles. *: p<0.05; **: p<0.01; ***: p<0.001 (Mann-Whitney-Wilcoxon test).

FIG. 2: In vitro production of cytokines in PBMC stimulated by LPS

Cytokines levels (IFN-γ, TNF-α and IL-4) were determined by X-Map assay in supernatants of cultured PBMC stimulated for 72 h with LPS (1 μg/mL). Box-and-whisker plots of log 10 (ratio of TH1/TH2) for each population: Community Dwelling (CD), Non Functional Decline (NFD) and Functional Decline (FD). Each box contains the middle 50% of the data, the horizontal line within each box represents the median, and the whiskers extend out to the 10th and 90th percentiles. *: p<0.05; ***: p<0.001 (Mann-Whitney-Wilcoxon test). A. Ratio of IFN-γ/IL-4 and B. Ratio of TNF-α/IL-4

FIG. 3: T cell subpopulations

Box-and-whisker plots for each population: Community Dwelling (CD), Non Functional Decline (NFD) and Functional Decline (FD). Each box contains the middle 50% of the data, the horizontal line within each box represents the median, and the whiskers extend out to the 10th and 90th percentiles. *: p<0.05; **: p<0.01 (Mann-Whitney-Wilcoxon test). A. Percentage of CD4; B: Percentage of CD8; C: CD4/CD8 ratio; D: Percentage of CD4+CD28+; E. Percentage of CD8+CD28+

FIG. 4: Telomere quantification

Box-and-whisker plots for each populations: Community Dwelling (CD), Non Functional Decline (NFD) and Functional Decline (FD). Each box contains the middle 50% of the data, the horizontal line within each box represents the median, and the whiskers extend out to the 10th and 90th percentiles. *: p<0.05 (Mann-Whitney-Wilcoxon test).

### DETAILED DESCRIPTION OF THE INVENTION

In the following passages, different aspects of the invention are described in more detail. Each aspect so described may be combined with ally other aspect or aspects unless clearly indicated to the contrary. In particular, any feature indicated as being preferred or advantageous may be combined with any other feature or features indicated as being preferred or advantageous.

In the context of the present invention, the terms used are to be construed in accordance with the following definitions, unless a context dictates otherwise. As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

Where embodiments are referred to as “comprising” particular features, elements or steps, this is intended to specifically include embodiments which consist of the listed features, elements or steps.

The present invention relates to methods for determining frailty and/or predicting functional decline. The term “frailty”, while lacking an official definition, is used herein to refer to a condition of biological aging, i.e. a reduced ability to function independently. Most importantly however, it is used herein as a predictor of a general decline in health. The frail face an immediate future of falls, deteriorating mobility, disability, hospitalization and death. More particularly, the frail are less likely to recover well from acute medical illness and hospitalization. Individuals that are not “frail” in the context of the present invention are referred to as “fit” or “robust”. Typically, the fit elderly are individuals, over years of age, living independently at home or in sheltered accommodation. They are freely ambulant and without significant hepatic, renal, cardiac, respiratory or metabolic disorder on either clinical examination or laboratory investigation. They do not receive regular prescribed medication.

Functional decline in a patient can be measured over a time period by determining the ability to perform specific tasks. Examples of how functional decline can be measured are provided herein. In particular embodiments, functional decline corresponds to a loss of at least 1 point on the ADL scale within a period of 3-month.

The methods of the present invention most particularly relate to predicting frailty or functional decline in elderly patients. The ability to identify an elderly at risk of functional decline allows targeted geriatric interventions which may prevent or limit functional loss.

The term “elderly” as used herein generally refer to individuals over 65 years of age. The methods of the invention thus, in particular embodiments are of interest in determining frailty in individuals over 65 years of age. In further particular embodiments, the methods of the invention are used to determine frailty in individuals of at least 70, most particularly at least 75 years of age.

The present inventors have identified methods for identifying patients which are frail or at risk of functional decline. It has been found that, for a similar illness, frail elder persons have differences in telomere length, differential gene and proteins expression, prolonged inflammatory activity and impaired immunological response, compared to a control group of non-frail elder persons. In particular, the measurement of T/S ratio was found to be indicative of frailty or of risk of functional decline. In that aspect, T corresponds to the PCR signal correlated with telomere length (whole telomere and/or telomeric repeats) and S corresponds to the PCR signal correlated with a reference single-copy gene. Examples of such single-copy genes include but are not limited to CD3, CD4, 36B4 (which encodes an acidic ribosomal phosphoprotein P0 (RPLP0)), ATP-synthase subunit 5B (A5B), Tumor Protein Translationally controlled 1 (TPT1), Signal Recognition Particle 14 kDa (SRP14), TATA-Binding Protein (TBP), Eukaryotic Elongation Factor 1A1 (EEF1A1), Hypoxanthine Phosphoribosyl-Transferase 1 (HPRT1), poly-Ubiquitin (Ubi), Glyceraldehyde-3-phosphate dehydrogenase (G3PD) and Beta-actin (ACTB). However suitable examples of single copy genes are known the skilled person.

In particular embodiments of the invention it is envisaged to normalize telomere measurement. The inventors herein provide a Method of normalization of telomere measurement by PCR:

Telomeres are repetitive sequence in genomic DNA. Their length, which may encompass thousands of base pairs, is related to their capacity of cellular replication. Indeed, this length diminishes by a number of telomeric repeats at each replication (approximately a few dozen of repeats). It was shown that cell lines present a limitative length which, when reached, marks the end of their replicative capacity (Hayflick's limit, linked to telomeric length by Alekseï Olovnikov). Stem cells and cancer cells are known to avoid this Hayflick's limit.

Measurements of telomeric length in heterogeneous cell populations present specific difficulties, notably related to the dispersion of telomeric length in these heterogeneous populations.

The use of the Polymerase Chain Reaction (PCR) has recently offered means to obtain a signal, called Telomere-to-Single-Copy Gene or T/S ratio, which is proportional to the average telomeric length in a cell population, whether this population is homogenous or not.

In order to avoid the dimerization of probes linked to the repetitive nature of the sequence, long non-complementary probes with sequences presenting from about 60% to about 95% homology with telomeric sequences, preferably from about 70% to about 90% homology with telomeric sequences, and more preferably from about 75% to about 85% homology with telomeric sequences, are used. Examples of probe sequences of the invention are: CGG-TTT-GTT-GGG-TTT-GGG-TTT-GGG-TTT-GGG-TT (SEQ ID NO: 1) and GGC-TTG-CCT-TAC-CCT-TAC-CCT-TAC-CCT (SEQ ID NO: 2).

Amplification leads to Amplicons of which the length and number are proportional to the initial telomere length. The intensity of SYBR green fluorescence is proportional to the Amplicons' number and length.

A specific problem linked to the evaluation of the T/S ratio is that it cannot be used with a classical calibrator to normalize results between laboratories, not even to ensure their reproducibility within the same laboratory. The reason is that a classical calibrator for quantitative PCR uses a target sequence in serial dilutions of concentration. This series of dilutions starting from a precisely known concentration is used to obtain a standard curve, allowing the association of a given signal intensity to a known concentration. The signal from an unknown sample is thus read against this curve, and its concentration in target sequences is deduced.

In some cases, when a standard of the target sequence, which can be a plasmid containing the target sequence, is not available, the measurement of multiple signals from a single sample can be a way of quantifying it, whereby at least one of the signals corresponds to a reporter or housekeeping gene. The concentration of this gene is postulated not to be influenced by external factors.

The measurement of the T/S ratio has similar technical constraints. However, the technical difficulty in case of the T/S ratio is specifically linked to the repetitive nature of the telomeric target sequences. The provision of a specific calibrator is required in order to obtain a T/S ratio value that can be normalized and give reproducible results, independently of the PCR machine, reagents and nature of the unknown samples used. This normalization is essential in the determination of a critical threshold of average telomere length, which can reflect several physiological phenomena, especially the prediction of senior frailty.

During a classical quantitative PCR, the resulting signal is dependent on the number of Amplicons produced by the PCR. The length of a specific Amplicon from a target sequence is the result of the choice of probes complementary to the sequence of interest. The signal generated by PCR follows an exponential curve. A critical threshold of detection is then selected in this exponential phase, which allows determining a Coefficient threshold (Ct). Starting from this Ct, the signal is postulated to be above the experimental background noise and related to the initial number of target sequence copies. For a given detection threshold, the Ct of an unknown sample thus depends only on the initial number of sequences of interest in the sample and the PCR efficiency. So, the Ct values can be plotted against the initial concentrations from a known standard (known by definition) containing the sequence of interest. This can be modelled in a semi-logarithmic function by an equation like Y=aX+B (quantification formula), where Y is the Ct, <<a>> varies with PCR efficiency, X is the number of copies of the sequence of interest in the standard and <<B>> corresponds to the Ct corresponding to the amplification of a single copy of the sequence of interest. Reporting the Y Ct value of a sample, which has an unknown X value, in this equation, allows deducing the x value. <<a>> and <<B>> parameters are postulated to be constant within a given experiment.

As indicated above the specific problem of telomere measurement comes from the repetitive nature of the target sequences. While the number of Amplicons is postulated to double at each cycle of a classical PCR, with the only correction of PCR efficiency, the number and the length of Amplicons during a quantitative PCR of telomeres will vary both according to the concentration and the number of telomeric repeats in the samples.

So instead of having:

APCRn=2⁽ⁿ⁾*i

where APCR is the number of Amplicons after n PCR cycles starting from i number of initial copies, we obtain in the case of repetitive sequences from a telomere amplification:

APCRn=2⁽ⁿ⁾*i′

where i′ is dependent both on the number and on the length of telomeres, as a unique telomere will not give 2 Amplicons (corrected for PCR efficiency), but instead:

APCRn=sum from 1 to m of i′

where, after n cycle of PCR (A_{PCRn}), m depends on the number of anchoring sites of the probes used. This number of anchoring sites varies with the length of the telomeres, which are determined by the number of telomeric repeats. After one cycle of PCR, we thus obtain several Amplicons of m different lengths. This multiplicity and heterogeneity of Amplicons starting from a sequence will be repeated and amplified with each PCR cycle.

The problem is further complicated by the absence of homogeneity in the initial telomere lengths in one sample, when coming from heterogeneous cell populations such as those found in a blood sample. If the PCR efficiency in a homogenous sample can be postulated to be constant, which allows the use of the quantification formula, this is not the case in a heterogeneous sample with various initial telomere lengths varying in unknown proportions. Indeed, the PCR efficiency is linked by a decreasing exponential function to the target sequence length duplicated in Amplicon. The parameters of this last exponential function vary according to the PCR machine, enzymes and reagents of the PCR, but are also dependant on factors such as endogenous inhibitors and facilitators specific to individual samples. In a classical quantitative PCR, this variation in efficiency is taken into account by the choice of a calibrator including the unique target sequence, and influences the “a” parameter of the quantification formula. The absence of calibrator taking into account the initial varying telomeric lengths does not allow making such an assumption in telomeres measurement by quantitative PCR as described above. The use of a probe anchoring to repetitive sites in this last kind of quantitative PCR results in PCR with various efficiencies depending on the number of repetitive sequences, and thus varying Amplicons lengths. This issue is addressed by the method described herein below.

If the expression of telomeric lengths as a T/S ratio allows reducing the problem caused by the absence of an external calibrator in the evaluation of the “a” parameter (dependent on the PCR machine and reagents), the T/S ratio does not permit reproducing the absolute values of signal from one laboratory to another, not even within the same laboratory at different time periods.

So, the establishment of a critical threshold, statistically associated with functional decline which can be relevant to seniors' frailty, demonstrates that a threshold of T/S ratio can be indicative of a risk of functional decline. Nevertheless, the absolute value of the T/S ratio could vary with the PCR machine and reagents used by each manipulator.

In order to answer this lack of normalization, the development of an external calibrator (“CalTel”) adapted to this specific use of quantitative PCR is essential. This calibrator's characteristics must integrate, in the <<a>> parameter of the quantification formula, the variations of efficiency linked to telomere lengths and those induced by the various lengths of resulting Amplicons at each polymerization cycle.

The inventors have developed a composite calibrator “CalTel” made of several standards containing various and known lengths of telomeric repeats and of known concentrations (T factor of the T/S ratio), together with a unique sequence of a single-copy gene corresponding to the S factor of the T/S ratio.

The use of this package of standards forms a calibrator that allows normalizing the lecture of a T/S signal between several users. The consequence is to make result transposable and reproducible, and to allow determining critical threshold on the “CalTel” basis, independently of the PCR machine and reagents used. This allows generalizing the senior's frailty prediction and their risk of functional decline on the basis of a normalized T/S ratio normalized by the “CalTel” calibrator.

The composite calibrator is a set of plasmids which can be described as follows: plasmid n° 1 containing one or more copy of the selected reference single-copy gene S and about 10 successive copies of the telomeric repeat TTAGGG, plasmid n° 2 containing one or more copy of the selected reference single-copy gene S and about 20 successive copies of the telomeric repeat TTAGGG, plasmid n° 3 containing one or more copy of the selected reference single-copy gene S and about 30 successive copies of the telomeric repeat TTAGGG, . . . , plasmid n° n containing one or more copy of the selected reference single-copy gene S and about 10×n successive copies of the telomeric repeat. TTAGGG. This composite calibrator will be used in PCR reactions with an appropriate amount of each of these plasmids, said amount being known or estimated from the total DNA quantity in the sample to be characterized. The amount of plasmids to be added should be in the same range as the amount of total DNA quantity in the sample. The PCR reactions will also contain primers specific to amplify the single-copy gene (S) and the telomeric repeats (T).

As a way of illustration, if a sample to be characterized contains an estimated 5·10⁵ cells (therefore 10⁶ copies of the reference single-gene copy S), “CalTel” could be set up as follows: a first PCR reaction containing a series of 10⁶ plasmids n° 1, a second PCR reaction containing 10⁶ plasmids n° 5, a third PCR reaction containing 10⁶ plasmids n° 10, a fourth PCR reaction containing 10⁶ plasmids n° 15, a fifth PCR reaction containing 10⁶ plasmids n° 20, a sixth PCR reaction containing 10⁶ plasmids n° 25, a seventh PCR reaction containing 10⁶ plasmids n° 30, . . . , a nth PCR reaction containing 10⁶ plasmids n° n would be performed . . . . This will allow constructing a reference curve of T/S signal directly related to a known telomere length of the plasmid inserts, where the T/S signal of the unknown sample can be reported. The resulting report in term of telomeric repeats of the composite calibrator is transposable and reproducible as it is independent of the PCR machine and PCR buffers and enzymes used.

### EXAMPLES

### Example 1

### Markers for Frailty and Functional Decline

Population Sample

The study was conducted at the University Hospital of Mont-Godinne (UCL, Belgium). The inventors considered three groups: young healthy volunteers, aged healthy volunteers and hospitalised aged patients. Young participants were recruited on a voluntary basis among personnel staff of the hospital Cliniques Universitaires de Mont-Godinne and with announcement in the hospital; they were 25 to 50 years, clinically symptom-free.

Aged participants were recruited on a voluntary basis from different seniors' associations. Eligible participants included respondents who: 1) were aged 75 years and over; 2) were not institutionalised; 3) had no evidence of an acute medical condition, nor deterioration of a chronic condition in the previous month. They were stratified to correspond to mean age and sex ratio of the expected hospitalised cohort. Eligible patients included subjects who were admitted through the emergency department, with one of the following diagnosis: 1) hip fracture after surgery; 2) decompensated heart failure; 3) documented infection (clinical, biological and at least bacteriological and/or radiological). The choice of three specific diseases allows easier comparisons and estimation of frailty modifications and represents different types of inflammatory challenges

General exclusion criteria were: use of anti-inflammatory drugs (including steroidal or nonsteroidal anti-inflammatory drugs) one week before the inclusion, or cancer. Specific exclusion criteria for hospitalized patients were: evaluation and biological sample impossible within the 96 hours of admission, requiring intensive care (impossibility of assessment), palliative care, already included, refusal of the patient or his referral, recent hospitalization in the previous two weeks (two-week premorbid evaluation non feasible), fully dependence on Activities of Daily Living (ADL) (no further functional decline possible), transfusion 24, hours previous blood samples.

Informed consent was obtained from the patient or from the caregiver for hospitalised patients who were unable to answer. The ethics committee of the hospital approved the study.

Data Collection

Healthy volunteers were evaluated the day of inclusion. For this hospitalised group, two points of evaluation were set: acute phase (defined as Day 2 to 4 after admission) and recovery phase (Day 9 to 15); this interval was chosen as sufficient to allow recovery of the acute stress and early evaluation of prognosis. Discharge evaluation took place the day before discharge. For some patients, the recovery and, the discharge evaluation were on the same day. Outcomes are assessed by phone contact at 3, 6 and 12 months after participation for aged volunteers and after discharge for hospitalised patients, by two skilled examiners. The 3-months interval was also chosen in others studies (Sager et al., 1996) as a optimal period allowing recovery with minimizing risk of new acute event.

Clinical Evaluation

Young patients were asked to report potential medication uses. Aged volunteers (N=33) had a structured geriatric examination the day of inclusion. Two trained interviewers collected information on demographic data and social support. Assessment of functional status was made with a modified Katz index of ADL (Katz S, Ford A B, Moskowitz R W, Jackson B A, Jaffe M W. Studies of Illness in the Aged. the Index of Adl: a Standardized Measure of Biological and Psychosocial Function. JAMA. 1963; 185:914-919.) and the Lawton scale for instrumental ADL (iADL) (Lawton M P, Brody E M. Assessment of older people: self-maintaining and instrumental activities of daily living. Gerontologist. 1969; 9:179-186.); the six ADL were quoted from 1 (independent) to 4 (fully dependant) (24-points) and the seven iADL from 1 (independent) to 0 (needing assistance) (7-points). Cognition was evaluated using a shortened form (21-points) of the Mini Mental State Examination (MMSE) for practical reasons as others studies (Cornette P, Swine C, Malhomme B, Gillet J-B, Meert P, D'Hoore W. Early evaluation of the risk of functional decline following hospitalization of older patients: development of a predictive tool. Eur J Public Health. 2006; 16:203-208.; Sager M A, Rudberg M A, Jalaluddin M et al. Hospital admission risk profile (HARP): identifying older patients at risk for functional decline following acute medical illness and hospitalization. J Am Geriatr Soc. 1996; 44:251-257.). Comorbidity was assessed with the Cumulative Rating Scale (CIRS). This scale was preferred to others burden of illness indexes as it has been adapted for to better reflect specificities of elderly persons (CIRS-G). Scoring was done using a manual of guidelines (Miller M D, Paradis C F, Houck P R et al. Rating chronic medical illness burden in geropsychiatric practice and research: application of the Cumulative Illness Rating Scale. Psychiatry Res. 1992; 41:237-248.). Self-rated health was treated as binary data (better/identical or poorer that persons of the same age). Nutrition was assessed by a shortened version of the Mini Nutritional Assessment (MNA). Other medical information collected were medication list, falls, hospitalisations and emergency admissions in the preceding year, alcohol and tobacco habits, recent vaccination, hearing or visual impairments. In addition, the interviewer recorded information on: grip strength (using a dynamometer), weight, size and Body Mass Index (BMI). Hospitalised patients were interviewed at admission with the same evaluation. Several scores predicting functional decline were completed: HARP (Sager et al., 1996), ISAR (McCusker J, Bellavance F, Cardin S, Trepanier S, Verdon J, Ardman O. Detection of older people at increased risk of adverse health outcomes after an emergency visit: the ISAR screening tool. J Am Geriatr Soc. 1999; 47:1229-1237.), SHERPA (Cornette et al., 2005). Functional status was also assessed at premorbid level (two weeks before admission).

Seven to ten days after inclusion, the investigators assessed the potential complications that occurred during the hospitalisation, evolution of the illness and evolution of grip strength. The current ability to perform the six ADL was assessed by interviewing the patient and the nursing staff. At discharge, the investigator noted discharges destination, possible modification of home health care, current ability to achieve the six ADL and the grip strength of the patient.

Outcomes

Primary outcome is functional decline (FD) from premorbid state to follow-up at 3 months. Functional decline was defined a priori as a increase of at least one point on the ADL scale (rated on 6) between premorbid evaluation on admission and the evaluation at 3, 6 and 12 months.

Secondary outcomes are: institutionalization, hospital readmission, emergency department visits, mortality, composite outcomes (FD plus institutionalization plus mortality) at 3, 6 and 12 months.

cDNA Expression Arrays

A customized EAT DualChip microarray allowing to assess the transcripts levels of 148 human genes was designed and produced by EAT (Eppendorf Array Technologies, Namur, Belgium).

Real-Time Quantitative Reverse Transcription-Polymerase

RT-qPCR experiments were performed in order to confirm expression levels. As expected, the expression profiles that were obtained with this technique are similar with those from DualChips experiments (correlation=0.94, data not shown). RT-qPCR was also performed in 93 samples in order to measure the expression of 4 other genes that are not represented onto the DualChips.

Statistical Analysis

Analyses were performed using R program. For comparison of qualitative data, the inventors used Fisher test between groups. For quantitative data, distribution was asymmetric so we used non parametric tests Mann-Whitney (two groups) and Kruskal-Wallis (three groups and more). Dynamic comparisons between acute and recovery phases was performed using Wilcoxon's test. Due to multiple analyses, the inventors presented p-value with the Benjamini correction (benjamini=pvaluexm/g, where m is the number of variables and g the rank of the variable according to p-value).

Pathway Analysis

The Benjamini score lower or equal to 0.05 defines the significance threshold. The gene expressions that had a significative difference were selected for pathway analysis using Ingenuity Pathway Analysis software (Ingenuity® System). The identified genes were mapped to biological networks available in the Ingenuity database and were then ranked by score. The score is the probability that a group of genes equal to or greater than the number in a network could be achieved by chance alone. A score of 2 have at least 99% confidence of not being generated by chance alone.

TABLE 1
Clinical characteristics of the participants

| | Community- | |
|---|---|---|
| | dwelling | Hospitalized |
| | seniors | patients |
| | N = 33 | N = 118 |
| Age (mean ± SD) | 82.3 ± 6.0 | 82.3 + 5.3 |
| Sexe (% female) | 58.0 | 60.2 |
| Marital status (N, %) | | |
| Married or in couple | 13 (39.4) | 50 (42.4) |
| Widow | 15 (45.5) | 58 (49.2) |
| Other | 5 (15.1) | 10 (8.47) |
| Living situation (N, %) | | |
| Home | 100 | 94 (79.7) |
| Nursing home | 0 | 24 (20.3) |
| Education >12 years (%) | 48.5 | 11.2 |
| Needs help at home (%) | 62.5 | 83.9 |
| Nb medic. (mean ± SD) | 3.2 ± 2.6 | 6.7 ± 3.1 |
| CIRS (mean ± SD) | | |
| No categories | 3.4 ± 1.9 | 5.9 ± 2.0 |
| Total score | 4.9 ± 3.7 | 13.4 ± 4.4 |
| Severity index | 1.34 ± 0.5 | 2.3 ± 0.4 |
| Hospitalisation in | 11 (30) | 36 (30.5) |
| the year N (%) | | |
| Fall in the year N(%) | 9 (27.3) | 82 (69.5) |
| Physical activity | | |
| Low N (%) | 33 (100) | 29 (24.6) |
| Moderate N (%) | 29 (87.9) | 54 (45.8) |
| Anxiety N (%) | 3 (9) | 50 (45.5) |
| Bad self perceived | 0 (0) | 17 (15.5) |
| health N (%) | | |
| ADL. mean ± SD | 6.3 ± 0.5 | 9.6 ± 4.3 |
| Median | 6.0 | 7.0 |
| IADL mean ± SD | 6.1 ± 1.3 | 3.3 ± 2.4 |
| Median | 7.0 | 3.0 |
| MMSE mean ± SD | 18.0 ± 2.3 | 14.9 ± 5.3 |
| median | 19.0 | 21.0 |
| <15* (N, %) | 1 (3) | 48 (40.7) |
| SHERPA (mean ± SD) | 3.0 ± 2.0 | 6.2 ± 2.9 |
| Categories N (%) | | |
| I | 19 (57.6) | 19 (16.1) |
| II | 8 (24.2) | 23 (19.5) |
| III | 4 (2.1) | 18 (15.3) |
| IV | 2 (6.1) | 58 (49.2) |

CIRS: Cumulative illness Rating Scale: measure of comorbidity (identifies 14 systems (e.g. heart, lung) which severity is scored from 1 to 4 (maximum). Total score reflects the sum of the severities for each system—maximum 56; no categories: no of impaired systems; severity index: mean severity (total score/no categories).
GDS: Geriatric Depression Scale—shortened form (screening tool for depression in geriatrics).
ADL: Activities of Daily Living (e.g. to wash, dress). Scores range from 6 (independent) to 24 (totally dependent).
IADL: Instrumental Activities of Daily Living (to drive, shop, cook). Scores range from 7 (independent) to 0 (totally dependent).
MMSE: Mini Mental Status Evaluation—shortened form. Evaluation of cognitive function. Scores range from 0 (maximum impairment) to 21 (normal cognitive function).
SHERPA: Score Hospitalier d'Evaluation du Risque de Perte d'Autonomie. This index was developed to predict functional decline (loss of independence in ADL) following hospitalization. The inventors used it as an indirect measure of frailty (a score of 5 or more indicates frailty). Scores range from 0 to11.5 (maximum risk of functional decline). Categories include increasing level of risk of functional decline: I, low; II, intermediate; III, moderate; IV: high.

Profiles of Biological Parameters Between Frail and Robust Patients

Frailty was defined according to the SHERPA score (if this was equal or higher than 5, the patient was considered as frail).

The frail group is composed of 44 patients and the robust group is of 78 patients. These two groups are comparable on the sex. However, a difference significantly on the age was observed (84.1 years for the frail group against 79.1 years for the robust group).

In Acute Phase

TABLE 2
Comparison of biological data in acute phase in
frail and robust patients

| | Patients | | | | | |
|---|---|---|---|---|---|---|
| | Robuts | Frail | | | | |
| Parameter | m ± SD | m ± SD | p.value | Benjamini | % | |
| IL10_NS_48 (pg/mL) | 2,536 ± 1,593 | 10,680 ± 15,077 | 1,898E−02 | 1,662E−01 | [symbol] | 24 |
| Neutrophils (nb/μL) | 6264,023 ± 2823,654 | 7779,141 ± 3806,132 | 1,135E−02 | 1,235E−01 | [symbol] | 81 |
| SjTREC | 35,457 ± 55,512 | 44,173 ± 209,048 | 1,141E−02 | 1,204E−01 | [symbol] | 80 |
| Albumin (mg/dL) | 3351,349 ± 518,383 | 3063,200 ± 548,334 | 5,788E−03 | 6,703E−02 | [symbol] | 109 |
| Creatin clearance | 56,589 ± 23,797 | 45,898 ± 23,122 | 1,228E−02 | 1,225E−01 | [symbol] | 123 |
| Haemoglobin (g/dL) | 12,173 ± 1,662 | 10,422 ± 1,792 | 1,332E−06 | 3,188E−05 | [symbol] | 117 |
| LDL cholesterol | 91,651 ± 26,522 | 80,711 ± 31,915 | 3,116E−02 | 2,542E−01 | [symbol] | 114 |
| Lymphocytes (nb/μL) | 1243,256 ± 400,140 | 1091,692 ± 466,931 | 2,343E−02 | 2,003E−01 | [symbol] | 114 |

* Variations are expressed in percent of the mean value of frail patients compared to robust patients ( [symbol] : decrease; [symbol] : increase).

TABLE 3
Gene expression in acute phase in frail and robust
patients

| | Old valid | Old fragile | | | | |
|---|---|---|---|---|---|---|
| Gene | m ± SD | m ± SD | p. value | Benjamini | % | * |
| ALDOA | 0,924 ± 0,322 | 1,051 ± 0,376 | 0,01470 | 0,13883 | [symbol] | 88 |
| CCNH | 0,732 ± 0,411 | 0,858 ± 0,400 | 0,00383 | 0,05290 | [symbol] | 85 |
| CTSD | 1,213 ± 0,443 | 1,382 ± 0,440 | 0,03410 | 0,26614 | [symbol] | 88 |
| IL1R2 | 1,754 ± 1,113 | 3,595 ± 1,667 | 0,00135 | 0,02195 | [symbol] | 49 |
| SOD2 | 1,149 ± 0,410 | 1,413 ± 0,538 | 0,00391 | 0,05015 | [symbol] | 81 |
| DNAJB1 | 0,885 ± 0,357 | 0,770 ± 0,397 | 0,01342 | 0,13016 | [symbol] | 115 |
| HSP9OAA1 | 1,337 ± 0,422 | 1,174 ± 0,456 | 0,04756 | 0,34145 | [symbol] | 114 |
| HSPA4 | 1,241 ± 0,470 | 1,079 ± 0,415 | 0,01822 | 0,16348 | [symbol] | 115 |

* Variations are expressed in percent of the mean value of frail patients compared to robust patients ( [symbol] : decrease; [symbol] : increase).

In Recovery Phase

TABLE 4
Comparison of biological data in recovery phase in
frail and robust patients

| | Patients | | | | | |
|---|---|---|---|---|---|---|
| | Robust | Frail | | | | |
| Parameter | m ± SD | m ± SD | p.value | Benjamini | % | * |
| Creatinin clearance | 56,556 ± 22,052 | 45,118 ± 20,853 | 1,096E−02 | 1,230E−01 | [symbol] | 125 |
| Haemoglobin (g/dL) | 11,787 ± 1,782 | 10,697 ± 1,419 | 1,922E−03 | 2,875E−02 | [symbol] | 110 |
| IFNg_S_72 (pg/mL) | 1539,562 ± 1589,442 | 795,097 ± 530,400 | 1,509E−02 | 1,389E−01 | [symbol] | 194 |
| Lymphocytes (nb/μL) | 1441,154 ± 571,988 | 1243,742 ± 440,735 | 3,641E−02 | 2,781E−01 | [symbol] | 116 |

* Variations are expressed in percent of the mean value of frail patients compared to robust patients ( [symbol] : decrease; [symbol] : increase).

TABLE 5
Gene expression in recovery phase in frail and
robust patients

| | Robust patients | Frail patients | | | | |
|---|---|---|---|---|---|---|
| Genes | m ± SD | m ± SD | p.value | Benjamini | % | * |
| FOS | 0,826 ± 0,254 | 0,934 ± 0,307 | 0,00356 | 0,05117 | [symbol] | 88 |
| HBEGF | 0,635 ± 1,306 | 1,265 ± 1,106 | 0,00073 | 0,01302 | [symbol] | 50 |
| IL1B | 0,471 ± 1,213 | 0,927 ± 1,253 | 0,00075 | 9,01276 | [symbol] | 51 |
| IL8 | 0,488 ± 1,145 | 0,984 ± 0,927 | 0,00003 | 0,00060 | [symbol] | 50 |
| PTGS2 | 0,431 ± 1,379 | 0,788 ± 1,112 | 0,00384 | 0,05110 | [symbol] | 55 |
| SOD2 | 1,141 ± 0,575 | 1,340 ± 0,451 | 0,04999 | 0,35190 | [symbol] | 85 |
| MYCBP | 1,135 ± 0,234 | 1,070 ± 0,236 | 0,03288 | 0,26230 | [symbol] | 106 |
| SRI | 1,183 ± 0,219 | 1,102 ± 0,229 | 0,03768 | 0,28179 | [symbol] | 107 |
| TNFRSF1A | 1,306 ± 0,405 | 1,151 ± 0,465 | 0,04306 | 0,31547 | [symbol] | 113 |

* Variations are expressed in percent of the mean value of frail patients compared to robust patients ( [symbol] : decrease; [symbol] : increase).

Profiles of Biological Parameters Between Patients with and without Functional Decline Three Months after Discharge

Functional decline is defined as loss of ability to perform basic self-care between the pre-hospitalization status and 3 months after discharge. Patient's with functional decline were significantly different in terms of age, living accommodation, falls in the year, independence in instrumental activities of daily living (e.g. to drive, to cook). They were more frequently cognitively impaired and the rate of functional decline was significantly higher in hip fracture.

Acute Phase

TABLE 6
Biological differences in acute phase according to
occurrence of functional decline

| | Decline 3 months after hospitalization | | | | | |
|---|---|---|---|---|---|---|
| | No | Yes | | | | |
| Parameter | m ± SD | m ± SD | p. value | Benjamini | % | * |
| IL6 (pg/mL) | 97,341 ± 90,080 | 169,141 ± 189,008 | 3,60^{E}−02 | 2,83^{E}−01 | [symbol] | 58 |
| CD4 | 47,006 ± 11,460 | 39,783 ± 11,935 | 1,82^{E}−03 | 5,06^{E}−02 | [symbol] | 118 |
| CD4/CD8 | 2,156 ± 1,126 | 1,597 ± 0,906 | 3,15^{E}−02 | 2,65^{E}−01 | [symbol] | 135 |
| CD4CD28 | 45,378 ± 11,599 | 37,109 ± 13,322 | 1,80^{E}−03 | 5,43^{E}−02 | [symbol] | 122 |
| Haemoglobin (g/dL) | 11,680 ± 1,964 | 10,352 ± 1,699 | 4,51^{E}−04 | 2,04^{E}−02 | [symbol] | 113 |
| IGF1 (pg/mL) | 225,151 ± 100,058 | 175,861 ± 73,978 | 1,72^{E}−02 | 2,07^{E}−01 | [symbol] | 128 |
| sjTREC | 70,991 ± 230,900 | 9,856 ± 12,782 | 2,47^{E}−02 | 2,48^{E}−01 | [symbol] | 720 |
| Telomeres length | 168,604 ± 207,158 | 79,489 ± 111,904 | 3,83^{E}−02 | 2,94^{E}−01 | [symbol] | 212 |

* Variations are expressed in percent of the mean value of patients with functional decline compared to patient without FD ( [symbol] : decrease; [symbol] : increase).

TABLE 7
Comparison of gene expression in acute phase
according to occurrence of functional decline

| | Decline 3 months after | | | | | |
|---|---|---|---|---|---|---|
| | hospitalization | | | | | |
| | No | Yes | | | | |
| Gene | m ± SD | m ± SD | p.value | Benjamini | % | * |
| SERPINB2 | 1,666 ± 1,321 | 2,779 ± 1,108 | 0,005 | 0,082 | [symbol] | 60 |
| IL1R1 | 1,146 ± 0,988 | 1,831 ± 0,858 | 0,003 | 0,058 | [symbol] | 63 |
| IL1RN | 1,002 ± 0,967 | 1,360 ± 0,930 | 0,045 | 0,312 | [symbol] | 74 |
| TIMP2 | 1,284 ± 0,749 | 1,661 ± 0,575 | 0,018 | 0,202 | [symbol] | 77 |
| MAP2K2 | 1,003 ± 0,359 | 1,138 ± 0,398 | 0,020 | 0,208 | [symbol] | 88 |
| GAPDH | 1,092 ± 0,229 | 1,159 ± 0,305 | 0,038 | 0,289 | [symbol] | 94 |
| MAPK9 | 0,908 ± 0,321 | 0,816 ± 0,348 | 0,039 | 0,288 | [symbol] | 111 |
| CASP8 | 0,754 ± 0,336 | 0,664 ± 0,365 | 0,030 | 0,258 | [symbol] | 114 |
| LCK | 0,667 ± 0,593 | 0,545 ± 0,645 | 0,029 | 0,259 | [symbol] | 122 |
| BCL2 | 0,708 ± 0,507 | 0,574 ± 0,701 | 0,050 | 0,331 | [symbol] | 123 |
| CD28 | 0,766 ± 0,699 | 0,594 ± 0,816 | 0,010 | 0,134 | [symbol] | 129 |

* Variations are expressed in percent of the mean value of patients with functional decline compared to patient without FD( [symbol] : decrease; [symbol] : increase).

Recovery Phase

TABLE 8
Biological differences in recovery phase according
to occurrence of functional decline

| | Decline 3 months after hospitalization | | | | | |
|---|---|---|---|---|---|---|
| | No | Yes | | | | |
| Parameter | m ± SD | m ± SD | p.value | Benjamini | % | * |
| CD8 | 25,397 ± 8,838 | 3,178 ± 10,033 | 4,83E−03 | 8,72E−02 | [symbol] | 77 |
| D-dimers (ng/mL) | 1835,163 ± 1335,193 | 2533,902 ± 1651,317 | 2,72E−02 | 2,59E−01 | [symbol] | 72 |
| CD4 | 48,041 ± 10,363 | 41,814 ± 10,543 | 5,81E−03 | 8,74E−02 | [symbol] | 115 |
| CD4/CD8 | 2,298 ± 1,448 | 1,478 ± 0,749 | 1,24E−02 | 1,60E−01 | [symbol] | 156 |
| CD4CD28 | 46,558 ± 9,696 | 38,973 ± 12,775 | 7,47E−03 | 1,08E−01 | [symbol] | 119 |
| Haemoglobin (g/dL) | 11,682 ± 1,756 | 10,507 ± 1,387 | 1,98E−03 | 5,10E−02 | [symbol] | 111 |

* Variations are expressed in percent of the mean value of patients with functional decline compared to patient without FD ( [symbol] : decrease; [symbol] : increase).

TABLE 9
Comparison of gene expression in recovery phase
according to occurrence of functional decline

| | Decline 3 months after | | | | | |
|---|---|---|---|---|---|---|
| | hospitalization | | | | | |
| | No | Yes | | | | |
| Gene | m ± SD | m ± SD | p.value | Benjamini | % | |
| SERPINB2 | 0,881 ± 1,230 | 1,657 ± 1,444 | 0,003 | 0,059 | [symbol] | 53 |
| IGFBP3 | 0,480 ± 1,097 | 0,814 ± 0,989 | 0,025 | 0,249 | [symbol] | 59 |
| TGFB1 | 1,061 ± 0,829 | 1,624 ± 0,951 | 0,004 | 0,090 | [symbol] | 65 |
| IL1R1 | 0,879 ± 1,234 | 1,197 ± 1,095 | 0,042 | 0,304 | [symbol] | 73 |
| IL15 | 0,994 ± 0,434 | 1,257 ± 0,523 | 0,001 | 0,055 | [symbol] | 79 |
| ILlORB | 1,079 ± 0,498 | 1,269 ± 0,429 | 0,045 | 0,318 | [symbol] | 85 |
| MDH1 | 0,869 ± 0,465 | 0,954 ± 0,655 | 0,046 | 0,313 | [symbol] | 91 |
| PSMA2 | 1,125 ± 0,206 | 1,027 ± 0,252 | 0,027 | 0,252 | [symbol] | 110 |
| CCL5 | 0,877 ± 0,329 | 0,762 ± 0,442 | 0,019 | 0,209 | [symbol] | 115 |
| PSMD11 | 1,143 ± 0,409 | 0,966 ± 0,501 | 0,028 | 0,253 | [symbol] | 118 |
| RPL13A | 0,883 ± 0,307 | 0,745 ± 0,427 | 0,001 | 0,050 | [symbol] | 119 |
| CASP8 | 0,821 ± 0,326 | 0,690 ± 0,418 | 0,002 | 0,050 | [symbol] | 119 |
| IL4R | 0,914 ± 0,491 | 0,742 ± 0,519 | 0,008 | 0,112 | [symbol] | 123 |
| DNAJB1 | 0,919 ± 0,479 | 0,742 ± 0,617 | 0,018 | 0,206 | [symbol] | 124 |
| CTSZ | 1,084 ± 0,853 | 0,776 ± 0,881 | 0,014 | 0,180 | [symbol] | 140 |

* Variations are expressed in percent of the mean value of patients with functional decline compared to patient without FD ( [symbol] : decrease; [symbol] : increase) .

Although the definition of frailty, as well as its physiopathology and characterisation may still be debated, outcomes of frailty are universally recognised: functional decline, disability, readmissions to hospital, geriatric syndromes (e.g. incontinence, delirium, fall, dementia), and mortality. The inventors used both an ‘a priori’ definition of frailty with the SHERPA score and an ‘a posteriori’ definition of frailty by measuring effective functional status three months after discharge. Patients with functional decline were considered as frail.

From tables 2 to 9, the inventors disclose a summarized list of biomarkers of frailty.

### Example 2

### Additional Markers for Predicting Functional Decline

Population Sample

In this study, participants were recruited among patients 75 years older and over hospitalized through the emergency department of a tertiary care university hospital. To reduce heterogeneity and address specific models of inflammatory challenges, the inventors selected patients with one of the following admission diagnoses: hip fracture after surgery, acute heart failure or documented infection (with bacteriological and/or radiological proofs). Patients were excluded if they used steroidal or nonsteroidal anti-inflammatory drugs one week before the inclusion, had cancer or a previous hospital stay within the 2 previous weeks, were admitted for intensive or palliative care or were completely dependent in activities of daily living (ADL). Informed consent was obtained from each participant or the caregiver when patients were unable to answer. The ethics committee of the hospital approved the study.

The data, including the clinical evaluation were collected as in Example 1.

Biological Variables

Blood sample were taken after 12-h overnight fast within the 96 hours of admission and were delivered to the central laboratory for measurement of C—reactive protein (CRP), white blood cells, urea, albumin, prealbumin and total cholesterol. Aliquots were stored at −80° C. until analysis. Interleukin-6 and IGF-1 were measured in duplicate using an enzyme-linked immunosorbent assay (ELISA) kits (Biosource, Nivelles, Belgium). The minimal detectable concentration for 11-6 and IGF-1 were respectively 2 pg/ml and 4.9 ng/ml.

Outcomes

Functional status was reassessed 3 months after discharge by phone contact and functional decline was defined as a loss of at least 1 point on the ADL scale between premorbid status (2 weeks before admission) and 3-month post-discharge evaluation.

Statistical Analysis

Comparisons between groups used the chi-square test for categorical variables and Wilcoxon rank sum test for numerical variables. Each of individual biological variables (CRP, white blood cells, urea, albumin, prealbumin, total cholesterol, 11-6 and IGF-1) was tested in a logistic regression model including SHERPA. The inventors assessed the accuracy of the predictive models in two ways. Discrimination—the ability to separate patients with and without disease—was measured by the area under receiver operating characteristic (ROC) curve. ROC curves were compared with the Hanley and McNeil method.

Calibration—the ability of the model to correctly estimate the probability of a future event—was measured by comparing the average predicted probability of the event in subgroups with the observed proportion developing the event within these subgroups. The inventors determined four subgroups using quartiles of estimated risk of functional decline (0 to <25%, 25 to <50%, 50 to <75%, 75 to 100%). The quality of calibration was assessed by computing the Brier score, for which 0 implies perfect prediction and 1 represent no predictive value (20).

Statistical analyses were performed by SPSS 15.0 software (SPSS Inc., Chicago, Ill.), and ROC curves were graphed by MedCalc 9.4 software (MedCalc, Mariakerke, Belgium).

Patient Characteristics

Hundred eighteen patients were included. Within 3 months of hospital discharge, 17 patients died. Since functional data at follow-up were missing for three patients, 98 patients were included in the analyses (mean age 81.8±5.2 years, 57% of females, 16 residents in nursing home). The distribution of the patients according to the three selected diagnoses was: 31 with heart failure, 32 with hip fracture and 35 with documented infection. Median length of stay was 12 days (interquartile range 8-19 days). Three months after discharge, functional decline was present in 46 patients (47%). Table 10 summarizes baseline characteristics of the patients according to the presence or absence of functional decline at 3 months. Patients with functional decline were 3 years older than those who maintained functional status (p=0.002), and had poorer preadmission functional status (ADL and IADL status). Rate of functional decline was significantly higher for hip fracture patients than for the other patients (75% versus 33%, p<0.001). There were no significant differences in gender or comorbidity. Median length of stay was significantly higher in patients who presented functional decline at follow-up (13 [10-24] versus 11 [7.5-17], p=0.046). In univariate analyses of biological markers, Il-6 and IGF-1 were significantly different according to further functional decline.

TABLE 10
Baseline characteristics of the study population

| | Functional decline | | |
|---|---|---|---|
| | 3 months after discharge | | |
| | Yes | No | |
| | n =46 | n =52 | p-value |
| Clinical characteristics | | | |
| Age (mean ± SD) | 83.6 ± 5.6 | 80.2 ± 4.3 | 0.002 |
| Sex (% females) | 65 | 50 | ns |
| Comorbidity (CIRS-G), mean ± SD | | | |
| Total score | 13.7 ± 4.4 | 12.2 ± 4.3 | ns |
| Severity index | 2.3 ± 0.3 | 2.3 ± 0.4 | ns |
| Primary medical diagnosis, n (%) | | | <0.001 |
| Hip fracture | 24 (75) | 8 (25) | |
| Heart failure | 11 (35) | 20 (65) | |
| Infection | 11 (31) | 24 (69) | |
| ADL, mean ± SD | 3.5 ± 2.1 | 4.4 ± 1.6 | 0.042 |
| IADL, mean ± SD | 2.9 ± 2.5 | 4.2 ± 2.2 | 0.009 |
| MMSE ( / 21), mean ± SD | 14.3 ± 4.7 | 16.7 ± 4.8 | 0.001 |
| SHERPA (0-11.5), mean ± SD | 7.1 ± 2.6 | 4.7 ± 2.7 | <0.001 |
| Biological values, median (interquartile range) | | | |
| CRP, mg/dl | 11.7 | 9.8 | ns |
| | (6.5-16.2) | (2.9-19.2) | |
| Leucocytes, nbr/microL | 8595 | 8615 | ns |
| | (7280-11780) | (6765-10165) | |
| Urea, mg/dl | 44 | 43 | ns |
| | (32-60) | (34.5-62) | |
| Clearance, ml/min | 46.6 | 50.0 | ns |
| | (32.1-68.1) | (34.4-72.3) | |
| Albumine, mg/dL | 3083 | 3347 | ns |
| | (2605-3401) | (2998-3595) | |
| Prealbumine, mg/dL | 13 | 14 | ns |
| | (10-14) | (11-20) | |
| I1-6, pg/ml | 98.6 | 60.4 | 0.019 |
| | (52.8-208.9) | (29.6-133.6) | |
| IGF-1, ng/ml | 155.5 | 204.2 | 0.002 |
| | (96.9-241.6) | (166.2-270.5) | |

CIRS-G: Cumulative Illness Rating Scale adapted for Geriatrics;
ADL: activities of daily living;
IADL: Instrumental ADL;
MMSE: Mini Mental State Examination;
SHERPA: “Score Hospitalier d'Evaluation du Risque de la Perte d′Autonomie”.

Multivariate Analyses

Using logistic regression, two biomarkers singled out: Il-6 and IGF-1. There was no correlation between these two parameters (spearman rho=−0.16).

SHERPA, Il-6 and IGF-1 were included in a combined predictive model (SHERPA+). Areas under the ROC curve and their confidence intervals [95% CI] were 0.79 [0.69-0.86] for SHERPA+versus 0.73 [0.63-0.81] for SHERPA alone. The logistic regression equation proposed for risk calculation is:

f=(SHERPA/4)+(Il-6/250)+(IGF-1/125)−0.45

Brier scores were 0.185 and 0.213 for SHERPA and SHERPA+ respectively, which emphasizes a good predictive capacity within individual patients. Table 11 illustrate the calibration of both models: with SHERPA+, mean predicted probability of functional decline was very close to the mean observed rate of decline within the four subgroups (determined by quartiles of estimated risk).

TABLE 11
Comparison of predicted risks and observed rates of functional decline

| | | SHERPA | | |
|---|---|---|---|---|
| | Probability of FD | Patients | Predicted FD | Actual FD |
| Subgroups | (%) | (n) | rate (%) | Rate (%) |
| 1 | 0-24 | 18 | 21 | 6 |
| 2 | 25-49 | 36 | 36 | 50 |
| 3 | 50-74 | 41 | 65 | 61 |
| 4 | 75-100 | 3 | 82 | 67 |
| | | SHERPA + (IL6 & IGF1) | | |
| | Probability of FD | Patients | Predicted FD | Actual FD |
| Subgroups | (%) | (n) | rate (%) | Rate (%) |
| 1 | 0-24 | 23 | 14 | 13 |
| 2 | 25-49 | 28 | 35 | 39 |
| 3 | 50-74 | 29 | 61 | 59 |
| 4 | 75-100 | 18 | 84 | 83 |

SHERPA: Score Hospitalier d'Evaluation du Risque de la Perte d′Autonomie;
SHERPA+: SHERPA+I1-6 & IGF-1;
FD: functional decline.
Subgroups are based on quartiles of estimated risk of FD; 4 categories were determined: 0 to 25%, 25% to <50%, 50% to <75% and 75% to 100%. Predicted FD rate is the mean predicted risk per subgroups, using the logistic model; actual FD rate is the presence of FD measured 3 months after discharge.

### Example 3

### Markers for Predicting Functional Decline

Participants were recruited among patients older than 75 years and over hospitalized through the emergency department of a tertiary care university hospital. To reduce heterogeneity and address specific models of inflammatory challenges, the inventors selected patients with three frequent acute conditions. Were eligible patients admitted through the emergency department with hip fracture, acute heart failure or documented infection. Patients were excluded if they could not be assessed within the 72 hours of admission (n=245). Other exclusion criteria were: use of anti-inflammatory drugs (n=110), cancer (n=60), admission for intensive or palliative care (n=53), previous hospitalization in the past two weeks (n=44), recent blood transfusion (n=10) and dependence for all ADL (n=7). Twenty-three patients (23/141) declined participation. In order to characterize stress-related differences, it was necessary to determinate reference immune profile of Community Dwelling (meaning those who are not in assisted living or nursing homes) seniors (75 years old and over, matched with mean age and sex ratio of the hospitalized group). They were recruited from different seniors' organizations and were free of acute medical condition or deterioration of a chronic condition in the previous month. Use of steroidal or nonsteroidal anti-inflammatory drugs one week before the inclusion was an exclusion factor for this group. Informed consent was obtained from each participant or the caregiver when patients were unable to answer. The ethics committee of the hospital approved the study.

The clinical assessment was performed as in Examples 1 and 2. The FD outcome was defined a priori as loss of at least one point on the ADL scale between premorbid status two weeks before admission and three-month post-discharge evaluation.

Isolation of PBMC and Culture

Blood sample were taken after 12-h overnight fast within the 96 hours of admission. PBMC were isolated from 16 ml of blood of healthy donors and patients by centrifugation on Ficoll-Hypaque gradient centrifugation (Becton Dickinson Vacutainer CPT). Cells were washed twice in HBSS and 10⁷ cells were stored at −80° C. for TREC analysis. For in vitro cytokine production, cells adjusted at 10⁶ cells/ml in a 96-wells plates were suspended in culture medium [RPMI-1640 10% FBS, penicillin-streptomycin (100 UI-100 μg/ml), HEPES, (0.5%), L-Glutamine (1 mM)]. Polyclonal stimulation of PBMC was performed in triplicate with PHA:LPS (0.5 μg/ml: 1 μg/ml). The plates were incubated at 37° C. in a humidified atmosphere containing 5% CO2 and 95% air during 72 hours. The supernatants were collected and used for cytokine quantification.

TREC Analyses

The inventors performed the quantification of sjTREC and DJβTREC, in order to quantify the thymic output and the intra-thymic proliferation. The protocol of TREC quantification has been fully described in Morrhaye, G.; Kermani, H.; Legros, J. J.; Baron, F.; Beguin, Y.; Moutschen, M.; Cheynier, R.; Martens, H. J.; Geenen, V. Impact of growth hormone (GH) deficiency and GH replacement upon thymus function in adult patients. PLoS One 2009, 4(5), e5668.

Multiplex Cytokine Assay

PBMC culture supernatants were analyzed for IL-4, IFN-γ, and TNF-αusing Bio-Plex human cytokine multiplex kits (500000068, BioRad, Ghent, Belgium). Calibration curves from recombinant cytokine standards were prepared by serial dilutions in the culture medium. Measurements were conducted according the manufacturer's instruction. The fluorescence intensity of the beads was measured using the Bio-Plex array reader (BioRad, Ghent, Belgium). Bio-Plex Manager software was used for data analysis.

Flow Cytometry

Cell phenotype was analyzed by flow cytometry with of a FACSCalibur cytometer and CellQuest software (BD Biosciences, Erembodegem, Belgium). Cells were stained with FITC-conjugated or PE-conjugated mAbs and APC-conjugated mAbs and PerCP-conjugated mAbs for the following surface markers: CD4, CD8, CD28 (BD Pharmingen).

Measurement of Relative Telomere Level

The measurement of “telomere length” was performed by real time kinetic quantitative PCR using a standard in the form of a plasmid encoding a single copy of CD3 (S). Telomere (T) PCRs and single-copy gene (S) PCR were always performed in duplicated measures of the T/S ratio. Two master mixes (IQ SYBR Green Supermix) of PCR reagents were prepared, one with the telomere primer pair (tel master mix), the other with the S primer pair (S master mix). The final concentrations of other reagents in the PCR were 0.2 mM each dNTP, 5 mM DTT and 1% DMSO). 20 μl of tel master mix or S master mix were added to each sample well and standard curve well of two separate plates. The final telomere primer concentrations were: tel-forward, 100 nM; tel-reverse, 300 nM. The final CD3 (single-copy gene) primer concentrations were: CD3-forward, 300 nM; CD3-reverse, 900 nM. The primer sequences were: tel-forward, CGG-TTT-GTT-GGG-TTT-GGG-TTT-GGG-TTT-GGG-TT (SEQ ID NO: 1); tel-reverse, GGC-TTG-CCT-TAC-CCT-TAC-CCT-TAC-CCT (SEQ ID NO: 2); CD3-forward, GGC-TGT-CCT-CAT-CCT-GGC-TA (SEQ ID NO: 3); CD3-reverse, TGA-CAG-AGG-TAG-GCT-GAA (SEQ ID NO: 4). For each standard curve, one reference DNA sample was diluted serially to produce five concentrations of DNA ranging from 2.5 pg to 2.75 ng/μl.

The thermal cycling profile for both amplicons began with 95° C. incubation for 30 s, then followed by 40 cycles of 95° C. for 15 s, 54° C. for 2 min for telomere amplification while for CD3 PCR, 30 s of 95° C. incubation was followed by 40 cycles of 95° C. for 15 s, 60° C. for 1 min. For each individual, the T/S ratio was assayed in duplicate and performed on a IcyclerIQ (Biorad).

Statistical Analysis

Comparisons between groups used the chi-square test for categorical variables and Mann-Whitney-Wilcoxon rank sum test for numerical variables. Univariate statistical analyses were performed on the Prism 4.0 program (GraphPad, San Diego, Calif.). The inventors used multiple logistic regression to compare the parameters of interest in patients with and without FD. To the baseline model, age, admission diagnosis and comorbidity were added. Each of the immunological markers was then entered in a logistic regression model including age, comorbidity score (CIRS-G total) and primary medical condition. Due to the limited number of patients, the factors were tested one by one. Statistical comparisons and logistic regression were performed by SPSS 15.0 software (SPSS Inc., Chicago, Ill.)

The inventors compared patients with and without FD (FD versus NFD).

### 3.1 Clinical Characterisation

One hundred eighteen participants were included among 670 patients. Within 3 months of hospital discharge, 17 patients died (14%). Since functional data at follow-up were missing for three patients, ninety-eight patients were finally included in the analyses (mean age 81.8±5.2 years, 57% of females). Patients were distributed in three diagnosis groups: heart failure (N=31), hip fracture (N=32) or infection (N=35). Three months after discharge, FD occurs in 46 patients. (47%). Table 12 summarizes baseline characteristics of hospitalized patients according to their functional evolution at three months. Patients with FD were significantly older, more dependent in ADL and more cognitively impaired. Hip fracture was the admission diagnosis most frequently associated with FD (75% versus 33% for infection and heart failure patients, p<0.001). There was no significant difference in comorbidity scores; as reflected by CIRS-G scores and number of medication. According to the method of recruitment, community-dwelling seniors (n=33) were matched for age and sex ratio, relatively healthy (CIRS-G total 4.9±3.7 versus 13.5±4.4, p<0.0001), cognitively and functionally intact (Table 12).

TABLE 12
Baseline characteristics of patients according to functional decline at 3 months

| | | FD in hospitalized | | |
|---|---|---|---|---|
| | Community- | patients | | |
| | dwelling | 3 months after discharge | | |
| | seniors | Yes (n = 46) | No (n = 52) | p-value* |
| Age (mean ± SD) | 82.3 ± 6.0 | 83.6 ± 5.6 | 80.2 ± 4.3 | 0.002 |
| Sex (% females) | 57.6 | 65.2 | 50.0 | NS |
| Comorbidity (CIRS), mean ± SD | | | | |
| Total score (0-56) | 4.9 ± 3.7 | 13.7 ± 4.4 | 12.2 ± 4.3 | NS |
| Severity index (0-4) | 1.3 ± 0.5 | 2.3 ± 0.3 | 2.3 ± 0.4 | NS |
| Number of medications, mean ± SD | 3.2 ± 2.6 | 7.2 ± 3.2 | 6.1 ± 2.7 | NS |
| Primary medical diagnosis, n (%) | NA | | | <0.001 |
| Hip fracture | | 24 (75) | 8 (25) | |
| Heart failure | | 11 (35) | 20 (65) | |
| Infection | | 11 (31) | 24 (69) | |
| ADL, mean ± SD (0-6) | 5.7 ± 0.5 | 3.5 ± 2.1 | 4.4 ± 1.6 | 0.042 |
| MMSE | 19.0 ± 2.3 | 14.3 ± 4.7 | 16.7 ± 4.8 | 0.001 |

Estimation of Thymic Function (sj and DJβTREC)

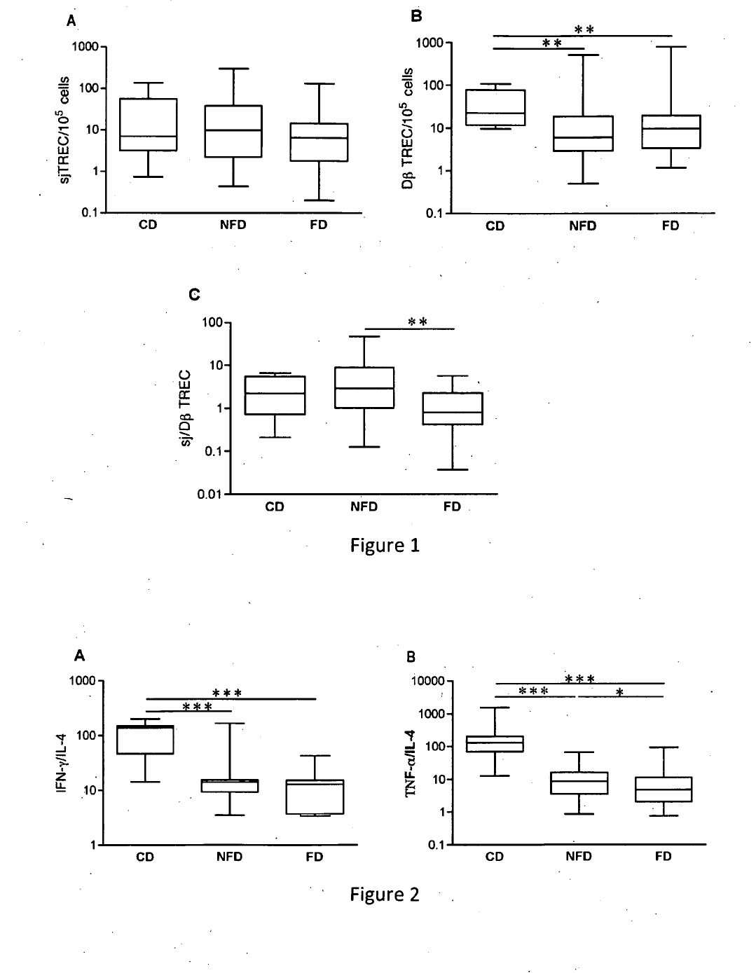

There was no significant difference in sjTREC levels between CD and hospitalized patients, neither comparing CD to NFD or FD. No significant difference could be observed between the two groups of hospitalized patients (FIG. 1a). Quantification of DJβTREC revealed a lower level in hospitalized patients than CD (p=0.0024) and comparison of CD with the NFD or FD groups revealed that the decrease of DJβTREC was critical for both (FIG. 1b).

Frequencies of sjTREC and DJβTREC were neither correlated with FD in hospitalized patients (FIGS. 1a, b). However, a significant decrease of the sj/DJβ TREC ratio was observed in patients with FD compared to NFD (p=0.009) (FIG. 1c). No difference appears between hospitalized patients as a whole and the CD.

The Balance Between Type 1 and Type 2 Cytokines

To test the hypothesis that ageing is associated with a shift in the balance between TH1 and TH2 lymphocytes, TNF-α/IL-4 or IFN-γ/IL-4 ratios were determined in stimulated PBMC. Compared to CD, both ratios were significantly decreased in patients (p<0.001), whenever they declined or not (FIGS. 2a and 2b). Nevertheless, TNFα/IL-4 ratio evidenced a significant difference between FD and NFD (p=0.047) (FIG. 2a).

Phenotype

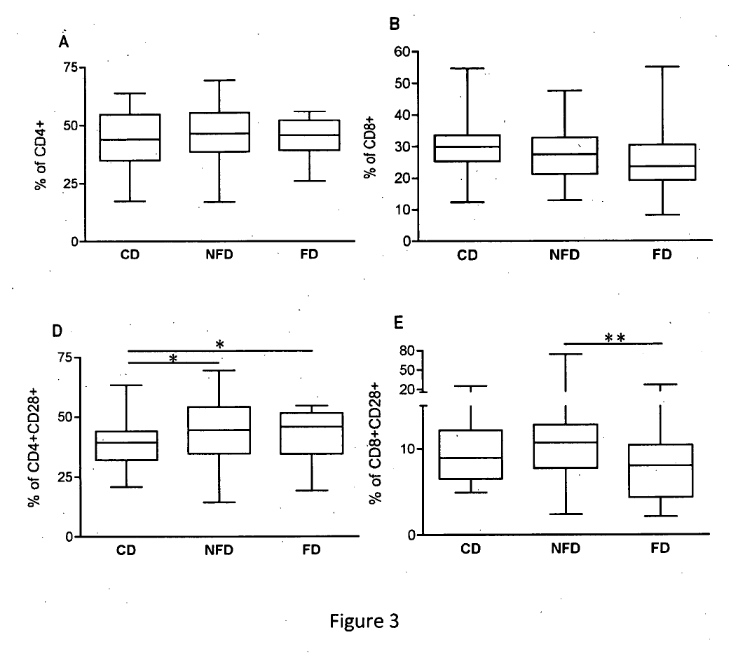

The inventors did not observed any difference of T cell subtypes between the studied groups regarding the CD4 or CD8 percentage (FIGS. 3a, b), but a significant difference of CD4/CD8 ratio (FIG. 3c) was observed between the control group of Community Dwellers (CD) and the FD group. A significant increase in the percentage of PBMC CD4+CD28+cells was detected in NFD or FD compared to CD (FIG. 3d). Of note, CD8+CD28+ T cells associated to FD were less abundant than their CD8+CD28+ counterpart in the NFD group (FIG. 3e).

Assessment of Telomere Length

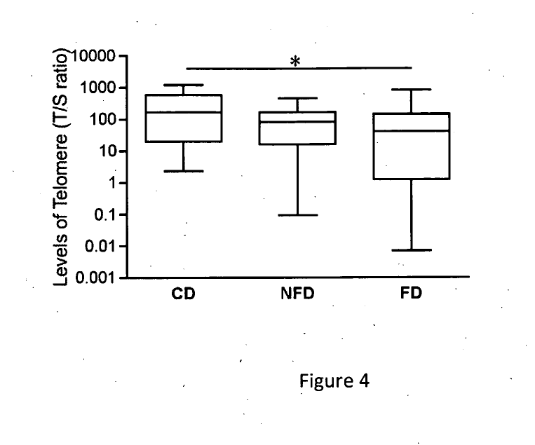

T/S ratio revealed that the telomere lengths present a significant decrease (p<0.05) between the CD and the patients with FD (FIG. 4).

Multivariate Analysis

To determine which variables were significantly associated with FD, the inventors created a logistic regression model where age admission diagnosis and comorbidity were significant predictors of FD. The model was tested by separated inclusion of each factor originally associated with FD in univariate analysis. Due to the limited numbers of patients for which all biological analyses were available, not all variables could be included at the same time. Only T/S ratio significantly improved the logistic regression model of FD based on age, admission diagnosis and CIRS-G.

Proteins of which the expression level is associated with frailty and/or Functional decline.

The inventors then compared the protein level of patients suffering or not of frailty and/or of functional decline. They measured significant differences between both populations. They conclude that one or several protein of the Table 13 represent(s) an additional predictive tool of frailty and/or of functional decline.

TABLE 13
identification of proteins over-expressed in frailty patients

| | Modif. Factor (negatif |
|---|---|
| | if overexpressed |
| Description | in frailaty patients) |
| Beta actin like protein 3 OS Homo sapiens GN ACTBL3 PE 1 SV 1 | −11.47 |
| POTE ankyrin domain family member F OS Homo sapiens GN POTEF PE 1 SV 2 | −7.46 |
| Glycogen phosphorylase liver form OS Homo sapiens GN PYGL PE 1 SV 4 | −4.06 |
| Glycogen phosphorylase brain form OS Homo sapiens GN PYGB PE 1 SV 5 | −3.29 |
| Keratin type I cytoskeletal 28 OS Homo sapiens GN KRT28 PE 1 SV 1 | −2.92 |
| CD5 antigen like OS Homo sapiens GN CD5L PE 1 SV 1 | −2.86 |
| Actin alpha skeletal muscle OS Homo sapiens GN ACTA1 PE 1 SV 1 | −2.80 |
| WD repeat containing protein 54 OS Homo sapiens GN WDR54 PE 1 SV 1 | −2.72 |
| Keratin type II cytoskeletal 6A OS Homo sapiens GN KRT6A PE 1 SV 3 | −2.08 |
| POTE ankyrin domain family member E OS Homo sapiens GN POTEE PE 1 SV 3 | −1.93 |
| Beta Ala His dipeptidase OS Homo sapiens GN CNDP1 PE 1 SV 3 | −1.67 |
| Complement C4 B OS Homo sapiens GN C4B PE 1 SV 1 | −1.52 |
| Transthyretin OS Homo sapiens GN TTR PE 1 SV 1 | −1.49 |
| Dihydropyrimidinase related protein 3 OS Homo sapiens GN DPYSL3 PE 1 SV 1 | −1.48 |
| Keratin type II cytoskeletal 6B OS Homo sapiens GN KRT6B PE 1 SV 5 | −1.40 |
| Keratin type II cytoskeletal 4 OS Homo sapiens GN KRT4 PE 1 SV 4 | −1.36 |
| Apolipoprotein D OS Homo sapiens GN APOD PE 1 SV 1 | −1.35 |
| Keratin type I cytoskeletal 26 OS Homo sapiens GN KRT26 PE 1 SV 2 | −1.34 |
| Zinc alpha 2 glycoprotein OS Homo sapiens GN AZGP1 PE 1 SV 1 | −1.32 |
| Mannose binding protein C OS Homo sapiens GN MBL2 PE 1 SV 2 | −1.31 |
| Complement factor D OS Homo sapiens GN CFD PE 1 SV 5 | −1.26 |
| Keratin type I cytoskeletal 24 OS Homo sapiens GN KRT24 PE 1 SV 1 | −1.26 |
| Complement C4 A OS Homo sapiens GN C4A PE 1 SV 1 | −1.23 |
| Keratin type I cytoskeletal 14 OS Homo sapiens GN KRT14 PE 1 SV 4 | −1.23 |
| Prostaglandin H2 D isomerase OS Homo sapiens GN PTGDS PE 1 SV 1 | −1.22 |
| Pregnancy zone protein OS Homo sapiens GN PZP PE 1 SV 3 | −1.19 |
| Keratin type I cytoskeletal 16 OS Homo sapiens GN KRT16 PE 1 SV 4 | −1.17 |
| Properdin OS Homo sapiens GN CFP PE 1 SV 2 | −1.17 |
| Complement C3 OS Homo sapiens GN C3 PE 1 SV 2 | −1.12 |
| Adiponectin OS Homo sapiens GN ADIPOQ PE 1 SV 1 | −1.11 |
| Protein S100 A9 OS Homo sapiens GN S100A9 PE 1 SV 1 | −1.09 |
| Ceruloplasmin OS Homo sapiens GN CP PE 1 SV 1 | −1.08 |
| C4b binding protein beta chain OS Homo sapiens GN C4BPB PE 1 SV 1 | −1.08 |
| Antithrombin III OS Homo sapiens GN SERPINC1 PE 1 SV 1 | −1.05 |
| Complement component C7 OS Homo sapiens GN C7 PE 1 SV 2 | −1.04 |
| Lumican OS Homo sapiens GN LUM PE 1 SV 2 | −1.04 |
| Coagulation factor IX OS Homo sapiens GN F9 PE 1 SV 2 | −1.03 |
| Tetranectin OS Homo sapiens GN CLEC3B PE 1 SV 2 | −1.03 |
| Alpha 1 antichymotrypsin OS Homo sapiens GN SERPINA3 PE 1 SV 2 | −1.02 |
| Hyaluronan binding protein 2 OS Homo sapiens GN HABP2 PE 1 SV 1 | −1.02 |
| Vitamin D binding protein OS Homo sapiens GN GC PE 1 SV 1 | −1.01 |
| Hemopexin OS Homo sapiens GN HPX PE 1 SV 2 | −1.01 |
| Afamin OS Homo sapiens GN AFM PE 1 SV 1 | −1.01 |
| Histidine rich glycoprotein OS Homo sapiens GN HRG PE 1 SV 1 | −1.01 |
| Complement component C8 beta chain OS Homo sapiens GN C8B PE 1 SV 3 | −1.01 |
| Prothrombin OS Homo sapiens GN F2 PE 1 SV 2 | 1.00 |
| Angiotensinogen OS Homo sapiens GN AGT PE 1 SV 1 | 1.00 |
| Coagulation factor X OS Homo sapiens GN F10 PE 1 SV 2 | 1.00 |
| Ribonuclease pancreatic OS Homo sapiens GN RNASE1 PE 1 SV 4 | 1.00 |
| Plasminogen OS Homo sapiens GN PLG PE 1 SV 2 | 1.02 |
| Vitamin K dependent protein S OS Homo sapiens GN PROS1 PE 1 SV 1 | 1.02 |
| Apolipoprotein C II OS Homo sapiens GN APOC2 PE 1 SV 1 | 1.02 |
| C reactive protein OS Homo sapiens GN CRP PE 1 SV 1 | 1.03 |
| Hepatocyte growth factor activator OS Homo sapiens GN HGFAC PE 1 SV 1 | 1.03 |
| Apolipoprotein A IV OS Homo sapiens GN APOA4 PE 1 SV 3 | 1.04 |
| Complement component C6 OS Homo sapiens GN C6 PE 1 SV 3 | 1.04 |
| Leucine rich alpha 2 glycoprotein OS Homo sapiens GN LRG1 PE 1 SV 2 | 1.04 |
| Attractin OS Homo sapiens GN ATRN PE 1 SV 2 | 1.04 |
| Hemoglobin subunit beta OS Homo sapiens GN HBB PE 1 SV 2 | 1.04 |
| Kininogen 1 OS Homo sapiens GN KNG1 PE 1 SV 2 | 1.05 |
| C4b binding protein alpha chain OS Homo sapiens GN C4BPA PE 1 SV 2 | 1.05 |
| Complement factor I OS Homo sapiens GN CFI PE 1 SV 1 | 1.05 |
| Extracellular matrix protein 1 OS Homo sapiens GN ECM1 PE 1 SV 2 | 1.05 |
| Keratin type I cytoskeletal 12 OS Homo sapiens GN KRT12 PE 1 SV 1 | 1.05 |
| Plasma protease C1 inhibitor OS Homo sapiens GN SERPING1 PE 1 SV 2 | 1.06 |
| Complement C2 OS Homo sapiens GN C2 PE 1 SV 2 | 1.06 |
| Complement factor H related protein 1 OS Homo sapiens GN CFHR1 PE 1 SV 2 | 1.06 |
| Ficolin 3 OS Homo sapiens GN FCN3 PE 1 SV 2 | 1.06 |
| Actin cytoplasmic 2 OS Homo sapiens GN ACTG1 PE 1 SV 1 | 1.06 |
| Beta 2 glycoprotein 1 OS Homo sapiens GN APOH PE 1 SV 3 | 1.07 |
| Keratin type II cytoskeletal 5 OS Homo sapiens GN KRT5 PE 1 SV 3 | 1.07 |
| Complement component C9 OS Homo sapiens GN C9 PE 1 SV 2 | 1.08 |
| Apolipoprotein C III OS Homo sapiens GN APOC3 PE 1 SV 1 | 1.08 |
| Galectin 3 binding protein OS Homo sapiens GN LGALS3BP PE 1 SV 1 | 1.08 |
| Complement C5 OS Homo sapiens GN C5 PE 1 SV 4 | 1.11 |
| Apolipoprotein E OS Homo sapiens GN APOE PE 1 SV 1 | 1.11 |
| L selectin OS Homo sapiens GN SELL PE 1 SV 2 | 1.11 |
| Kallistatin OS Homo sapiens GN SERPINA4 PE 1 SV 3 | 1.12 |
| Alpha 1B glycoprotein OS Homo sapiens GN A1BG PE 1 SV 3 | 1.13 |
| Alpha 2 HS glycoprotein OS Homo sapiens GN AHSG PE 1 SV 1 | 1.13 |
| Complement factor B OS Homo sapiens GN CFB PE 1 SV 2 | 1.14 |
| Serum amyloid P component OS Homo sapiens GN APCS PE 1 SV 2 | 1.14 |
| Carboxypeptidase N catalytic chain OS Homo sapiens GN CPN1 PE 1 SV 1 | 1.14 |
| Actin cytoplasmic 1 OS Homo sapiens GN ACTB PE 1 SV 1 | 1.15 |
| Mannan binding lectin serine protease 1 OS Homo sapiens GN MASP1 PE 1 SV 3 | 1.15 |
| Vitronectin OS Homo sapiens GN VTN PE 1 SV 1 | 1.16 |
| N acetylmuramoyl L alanine amidase OS Homo sapiens GN PGLYRP2 PE 1 SV 1 | 1.16 |
| Complement component C8 alpha chain OS Homo sapiens GN C8A PE 1 SV 2 | 1.16 |
| Biotinidase OS Homo sapiens GN BTD PE 1 SV 2 | 1.16 |
| Pigment epithelium derived factor OS Homo sapiens GN SERPINF1 PE 1 SV 3 | 1.17 |
| Heparin cofactor 2 OS Homo sapiens GN SERPIND1 PE 1 SV 3 | 1.17 |
| Glutathione peroxidase 3 OS Homo sapiens GN GPX3 PE 1 SV 2 | 1.17 |
| Protein AMBP OS Homo sapiens GN AMBP PE 1 SV 1 | 1.19 |
| Insulin like growth factor binding protein complex acid labile chain OS Homo | 1.19 |
| sapiens GN IGFALS PE 1 | |
| Carboxypeptidase N subunit 2 OS Homo sapiens GN CPN2 PE 1 SV 2 | 1.19 |
| Complement component C8 gamma chain OS Homo sapiens GN C8G PE 1 SV 3 | 1.20 |
| Alpha 2 antiplasmin OS Homo sapiens GN SERPINF2 PE 1 SV 3 | 1.21 |
| Complement factor H OS Homo sapiens GN CFH PE 1 SV 4 | 1.22 |
| Retinol binding protein 4 OS Homo sapiens GN RBP4 PE 1 SV 3 | 1.22 |
| Serum paraoxonase arylesterase 1 OS Homo sapiens GN PON1 PE 1 SV 2 | 1.22 |
| Complement C1r subcomponent OS Homo sapiens GN C1R PE 1 SV 2 | 1.25 |
| Clusterin OS Homo sapiens GN CLU PE 1 SV 1 | 1.25 |
| Thyroxine binding globulin OS Homo sapiens GN SERPINA7 PE 1 SV 2 | 1.25 |
| Plasma kallikrein OS Homo sapiens GN KLKB1 PE 1 SV 1 | 1.25 |
| Complement C1s subcomponent OS Homo sapiens GN CIS PE 1 SV 1 | 1.26 |
| Coagulation factor XII OS Homo sapiens GN F12 PE 1 SV 2 | 1.26 |
| Complement factor H related protein 2 OS Homo sapiens GN CFHR2 PE 1 SV 1 | 1.26 |
| Cholinesterase OS Homo sapiens GN BCHE PE 1 SV 1 | 1.26 |
| Inter alpha trypsin inhibitor heavy chain H4 OS Homo sapiens GN ITIH4 PE 1 SV 4 | 1.28 |
| Inter alpha trypsin inhibitor heavy chain H3 OS Homo sapiens GN ITIH3 PE 1 SV 2 | 1.28 |
| Keratin type I cytoskeletal 27 OS Homo sapiens GN KRT27 PE 1 SV 1 | 1.32 |
| Complement C1q subcomponent subunit C OS Homo sapiens GN C1QC PE 1 SV 3 | 1.34 |
| Complement C1q subcomponent subunit B OS Homo sapiens GN C1QB PE 1 SV 2 | 1.34 |
| Carboxypeptidase B2 OS Homo sapiens GN CPB2 PE 1 SV 1 | 1.34 |
| Inter alpha trypsin inhibitor heavy chain H1 OS Homo sapiens GN ITIH1 PE 1 SV 3 | 1.35 |
| Gelsolin OS Homo sapiens GN GSN PE 1 SV 1 | 1.36 |
| Keratin type I cytoskeletal 17 OS Homo sapiens GN KRT17 PE 1 SV 2 | 1.38 |
| Inter alpha trypsin inhibitor heavy chain H2 OS Homo sapiens GN ITIH2 PE 1 SV 2 | 1.39 |
| Glycogen phosphorylase muscle form OS Homo sapiens GN PYGM PE 1 SV 6 | 1.40 |
| Keratin type II cytoskeletal 2 epidermal OS Homo sapiens GN KRT2 PE 1 SV 2 | 1.42 |
| Keratin type I cytoskeletal 10 OS Homo sapiens GN KRT10 PE 1 SV 5 | 1.43 |
| Keratin type I cytoskeletal 25 OS Homo sapiens GN KRT25 PE 1 SV 1 | 1.46 |
| Keratin type I cytoskeletal 15 OS Homo sapiens GN KRT15 PE 1 SV 2 | 1.48 |
| Keratin type II cytoskeletal 1 OS Homo sapiens GN KRT1 PE 1 SV 6 | 1.62 |
| Keratin type I cytoskeletal 9 OS Homo sapiens GN KRT9 PE 1 SV 3 | 2.01 |
| Beta actin like protein 2 OS Homo sapiens GN ACTBL2 PE 1 SV 2 | 2.36 |
| Actin gamma enteric smooth muscle OS Homo sapiens GN ACTG2 PE 1 SV 1 | 2.72 |
| Keratin type I cytoskeletal 13 OS Homo sapiens GN KRT13 PE 1 SV 3 | 6.82 |

1. A method to predict the functional decline of a patient comprising the steps of
measuring the telomere length of PBMC from a blood sample obtained from said patient; and
deducing from said telomere length whether said patient is likely to have a functional decline.

2-3. (canceled)

4. The method according to claim 1, wherein a reduced telomere length represents a worse prognosis.

5. The method according to claim 1, wherein the measurement of telomere length is calibrated by a method comprising the steps of:
amplifying and quantifying the complete telomere using specific primers;
amplifying and quantifying a reference single-copy gene (S) in the same sample; and
comparing said length with a standard comprising 1, 2, 3, 4 or more DNA fragments (possibly (each) on a plasmid) (each) in a known concentration encoding one single-copy gene (S) (or fragment thereof) and several telomeric repeats of different sizes (T).

6. The method according to claim 1, which further comprises one or more of the steps selected from:
determining the functional status of said patient;
measuring cytokine content of the said blood sample and/or cytokine produced by PBMC present in said blood sample; and
measuring sj and djβ TREC in PBMC present in the said blood sample.

7. The method according to claim 6, wherein the cytokines are 1, 2, 3, 4 or all the cytokines selected from the group consisting of TNFα, IFNγ, IL4, IL-6 and IGF1.

8-20. (canceled)

21. A kit comprising
means to measure telomere length;
optionally means to measure sjTREC and DβTREC;
optionally means to measure cytokine content;
optionally means to measure mRNA content.

22. The kit of claim 21, wherein the means to measure the telomere length are specific primers able to amplify by PCR the whole telomere and a single-copy gene and, optionally, buffers and reagent for quantitative PCR, preferably Real-Time quantitative PCR.

23. The kit according to claim 21, wherein the means to measure cytokine content comprise antibodies specifically recognizing said cytokines.

24. (canceled)

25. The kit according to claim 21, wherein the cytokines are 1, 2, 3, 4 or all the cytokines selected from the group consisting of TNFα, IFNγ IL4, IL-6 and IGF1.

26-27. (canceled)

28. The kit according to claim 21, wherein the means to measure telomere length comprise:
primers and possibly buffer to amplify and possibly to quantify the complete (whole) telomere;
specific primers and possibly buffer to amplify and possibly to quantify a single-copy gene;
1, 2, 3, 4 or more DNA fragments (possibly (each) on a plasmid) (each) in a known concentration encoding one or more single-copy gene (S) (or fragment thereof) and several telomeric repeats of different size (T).

29. A method for calibrating the measurement of telomere length comprising the step of:
amplifying and quantifying the complete telomere using specific primers;
amplifying and quantifying a reference single-copy gene (S) in the same sample; and
comparing said length with a standard comprising 1, 2, 3, 4 or more DNA fragments, optionally each provided on a plasmid, (each) in a known concentration encoding one single-copy gene (S) (or fragment thereof) and several telomeric repeats of different sizes (T).

30. A method for determining the effect of a compound on the risk of functional decline of a patient comprising the steps of:
Measuring the telomere length of PBMC from a blood sample obtained from a patient prior to administration of a compound of interest;
Measuring the telomere length of PBMC from a blood sample obtained from said patient after administration of said compound of interest; and
Comparing the values of telomere length and deducing therefrom whether the compound has an effect on the risk of functional decline of said patient.
